(19) United States
(12) Patent Application Publication
(10) Pub. No.: US 2022/0185810 A1
(43) Pub. Date: Jun. 16, 2022

(54) Novel Process for the Preparation of Filgotinib and Intermediates Thereof

(71) Applicant: UNICHEM LABORATORIES LIMITED, Mumbai (IN)

(72) Inventors: Dhananjay G. Sathe, Thane (IN); Arijit Das, Porvorim (IN); Bhavesh Patel, Navsari (IN); Eknath Kshirsagar, Solapur (IN); Dipak Patil, Dhule (IN); Ashok Matale, Nashik (IN)

(21) Appl. No.: 17/599,642

(22) PCT Filed: Mar. 28, 2020
(86) PCT No.: PCT/IB2020/052977

§ 371 Date: Sep. 29, 2021

(30) Foreign Application Priority Data
Mar. 30, 2019 (IN) 201921012919

(51) Int. Cl. C07D 471/04; C07D 213/75

(52) U.S. Cl. CPC C07D 471/04; C07D 213/75

(57) ABSTRACT

The present invention relates to a novel process for the preparation of filgotinib or a pharmaceutically acceptable salt and intermediates thereof which avoid Suzuki coupling reaction.

### FIELD OF THE INVENTION

The present invention is related to a novel process for the preparation of Filgotinib or its salts and its intermediates thereof.

### BACKGROUND OF THE INVENTION

Filgotinib is a highly selective JAK1 inhibitor, discovered and developed by Galapagos for the treatment of rheumatoid arthritis, Crohn's disease and ulcerative colitis (UC).

Chemically filgotinib is described as Cyclopropanecarboxylic acid {5-[4-(1,1-dioxo-1-thiomorpholin-4-ylmethyl)-phenyl]-[1,2,4]triazolo[1,5-a]pyridin-2-yl}-amide or N-[5-[4-[(1,1-dioxo-1,4-thiazinan-4-yl) methyl] phenyl]-[1,2,4]-triazolo[1,5-a]pyridin-2-yl]cyclopropane carboxamide having compound of formula (I).

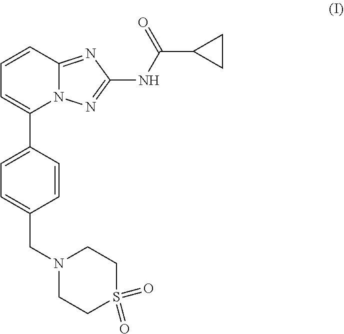 [SMILES: O=C(Nc1nc2cccc(-c3ccc(CN4CCS(=O)(=O)CC4)cc3)n2n1)C1CC1]

Filgotinib and a process for its preparation is disclosed in U.S. Pat. No. 8,088,764. The process therein involves several approaches for the preparation of Filgotinib as follows:

Approach I: Condensing compound 2 with compound 3 followed by cyclization using hydroxyl amine hydrochloride and then amidation with compound 6 to obtain compound 7.
Filgotinib compound of formula (I) is prepared by Suzuki coupling of compound 7 with compound 8 in presence of PdCl₂dppf and K₂CO₃.
Approach II: Compound 10 is obtained by Suzuki coupling of compound 7 with compound 9 in presence of PdCl₂dppf and K₂CO₃ followed by bromination to obtain compound 11 and its condensation with compound 12 to obtain filgotinib compound of formula (I).

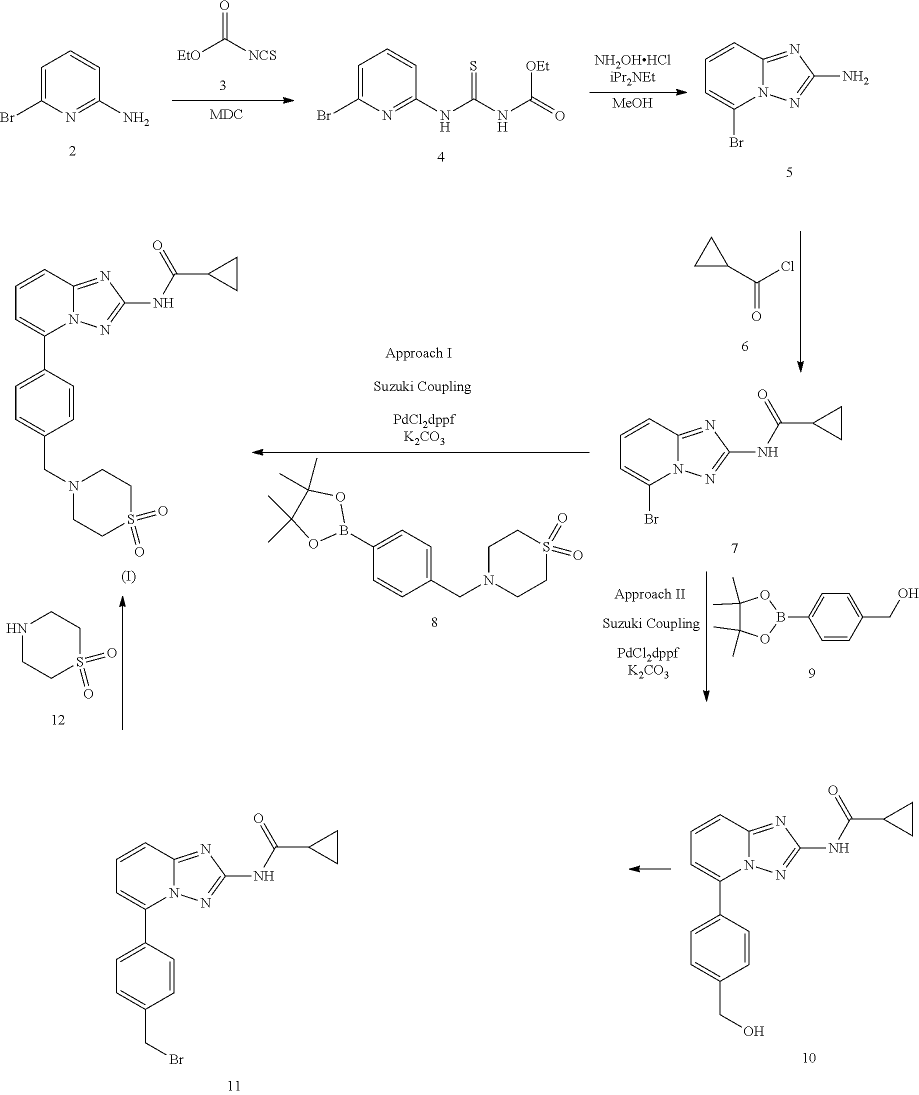

Approach III: Aldehyde compound 14 is prepared via Suzuki coupling of Boronic acid compound 13 with compound 7 in presence of PdCl₂dppf; resulting compound 14 is treated with thiomorpholin 1, 1-dioxide compound 12 in presence of Na(CN)BH₃ and Ti(Opr)₄ to obtained filgotinib compound of formula (I).

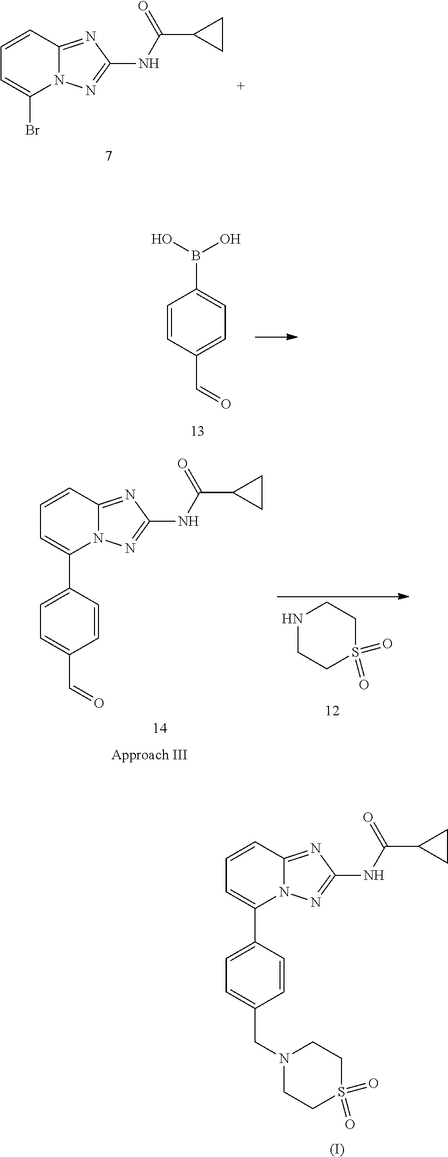

CN104987333B discloses different approach for the preparation of filgotinib, in which compound 15 is treated with di-tert-butyl carbamate followed by hydrolysis to obtain compound 16, which is then treated with trifluoro methane sulfonic anyhydride to obtain compound 17. Compound 18 is prepare by Suzuki coupling of compound 17 with compound 8 in presence of PdCl₂(PPh₃)₂ followed by deprotection to obtain compound 18; which is then reacted with compound 3 to obtain compound 19 followed by cyclization with hydroxy amine hydrochloride to obtain compound 20. Amidation of Compound 20 is carried out with cyclopropanecarbonyl chloride compound 6 to obtain Filgotinib compound of formula (I).

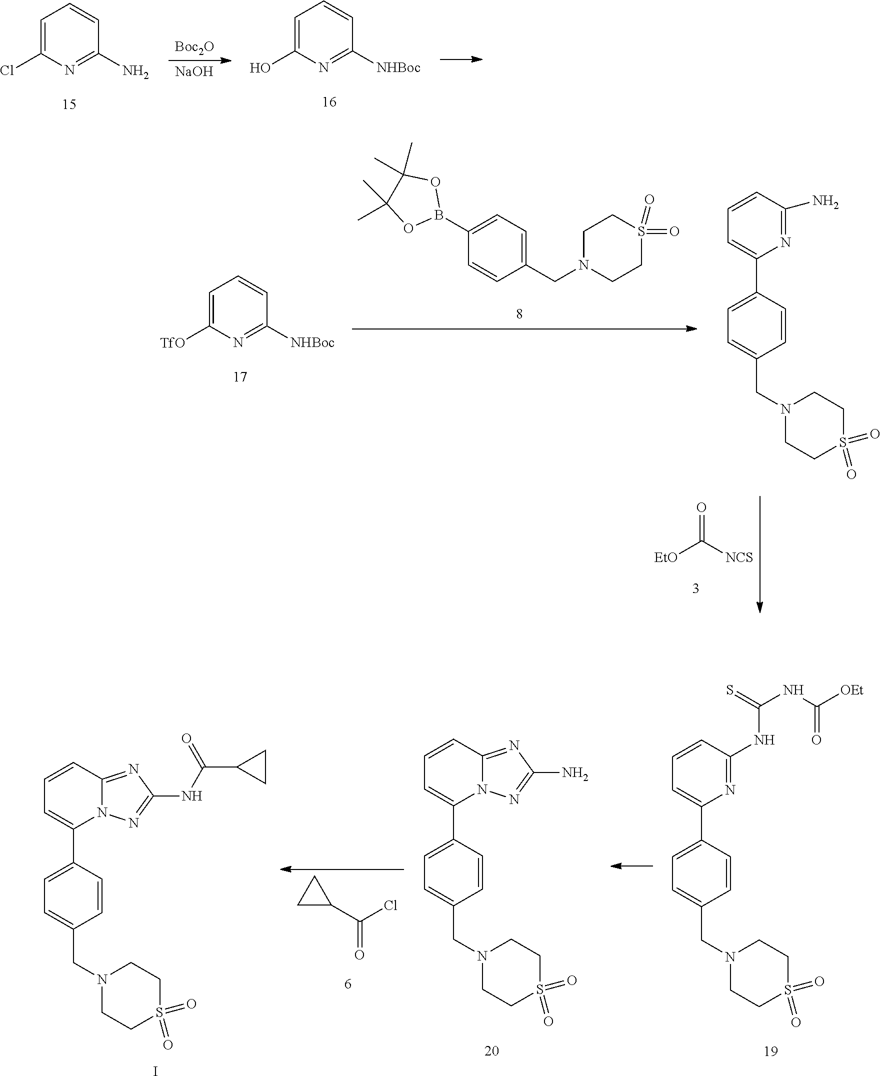

Prior art references such as (US′764, CN′333) discloses Suzuki coupling reaction which involve use of organoborane compounds and PdCl₂dppf as a catalyst, moreover US′764 also discloses reductive alkylation by using Na(CN)BH₃ and Ti(Opr)₄ during the preparation of filgotinib.

The catalysts used in the above prior art references are very expensive and difficult to recover and reuse. These processes are considered complex, uneconomical and time consuming, hence not suitable for commercial production.

It is, therefore, desirable to provide an efficient process for the preparation of filgotinib which avoid the use of Suzuki coupling, reductive alkylation and improves the economics by employing less expensive reagents and is more productive.

Present invention include less hazardous and environmentally friendly reagents, reduced cost, greater simplicity, increased product purity and increased yield of the product.

### OBJECT OF THE INVENTION

In order to overcome the defects in the prior art, the present invention provides an efficient method for preparing filgotinib compound of formula (I) or salts thereof which is suitable for industrial production.

Yet another object of the invention is to provide the novel process for the preparation of filgotinib, its salts and intermediate thereof, which avoid Suzuki coupling reaction.

Yet another object of the present invention is to provide novel intermediates for preparation of filgotinib or a pharmaceutically acceptable salt thereof.

### SUMMARY OF THE INVENTION

The present invention provides a novel process for the preparation of filgotinib compound of formula (I) or salts thereof comprising steps of;
a) condensing compound of formula (II) or salt thereof with compound of formula (III) to obtain compound of formula (IV); wherein R is C₁-C₄ alkyl group;
b) cyclizing compound of formula (IV) using hydroxyl amine or its acid additional salt to obtain compound of formula (V);
c) amidation of compound of formula (V) with cyclopropanecarbonyl chloride to obtain compound of formula (VI);
d) treating compound of formula (V) with halogenating agent to obtain compound of formula (VI);
e) condensing compound of formula (VI) with thiomorpholine-1,1-dioxide to obtain compound of formula (I);
f) optionally converting compound of formula (I) into its salt.

Yet another object of the present invention is to provide a novel process for the preparation of intermediates useful for preparation of filgotinib or salts thereof.

Yet another object of the present invention is to provide novel intermediates compound of formula (IV), (V) or its salt thereof.

Yet another object of the present invention is to provide a novel process to prepare intermediates compound of formula (IV), (V) or its salt thereof useful for preparation of filgotinib or salts thereof.

In one aspect, the present invention relates to a process for preparation of compound of formula (I), or a pharmaceutically acceptable salt thereof, comprising the steps of:

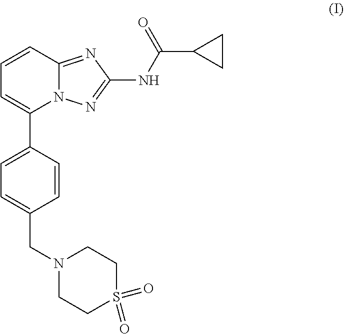 [SMILES: O=C(Nc1nc2cccc(-c3ccc(CN4CCS(=O)(=O)CC4)cc3)n2n1)C1CC1]

a) condensing a compound of formula (II) or salt thereof with a compound of formula (III) in organic solvent to obtain a compound of formula (IV); wherein R is C₁-C₄ alkyl;

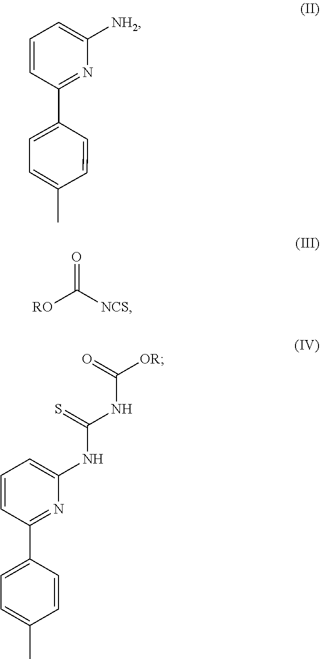

b) cyclizing the compound of formula (IV) using hydroxyl amine or its acid additional salt in presence of a solvent and a base to obtain a compound of formula (V) or a salt thereof;

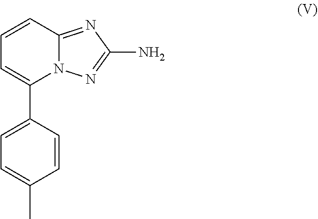 [SMILES: Cc1ccc(-c2cccc3nc(N)nn23)cc1]

c) reacting the compound of formula (V) with cyclopropanecarbonyl chloride in presence of a solvent and a base to obtain a compound of formula (VI);

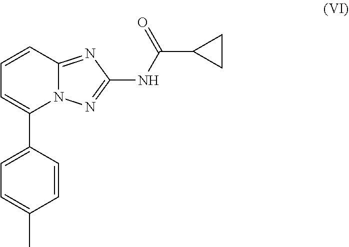 [SMILES: Cc1ccc(-c2cccc3nc(NC(=O)C4CC4)nn23)cc1]

d) treating the compound of formula (VI) with halogenating agent in presence of a solvent to obtain a compound of formula (VII);

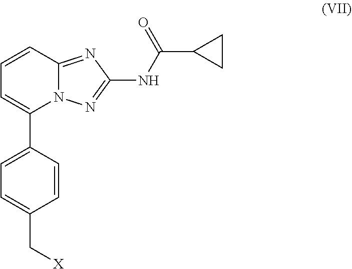

wherein, X is Cl, Br, I or F;
e) condensing the compound of formula (VII) with thiomorpholine-1,1-dioxide in presence of organic solvent to obtain a compound of formula (I); and
f) optionally converting the compound of formula (I) into its pharmaceutically acceptable salt.

In another aspect of the present invention, the above said process is shown in the following General Scheme.

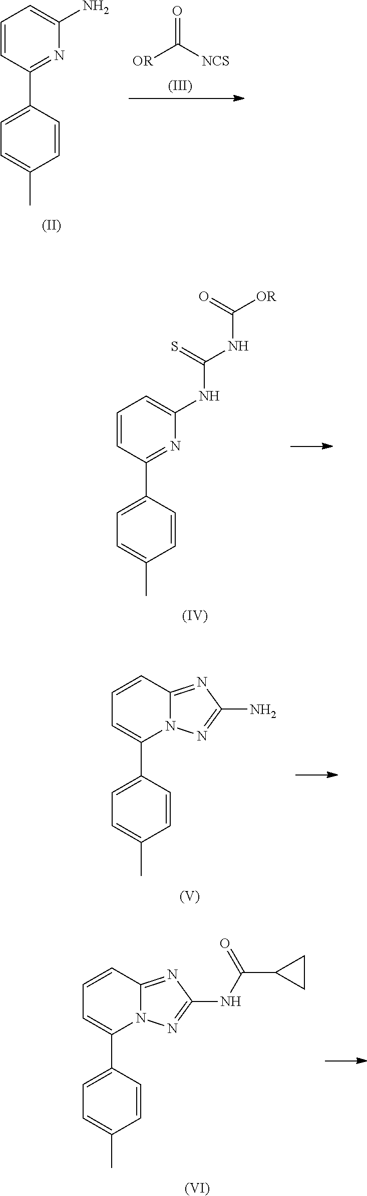

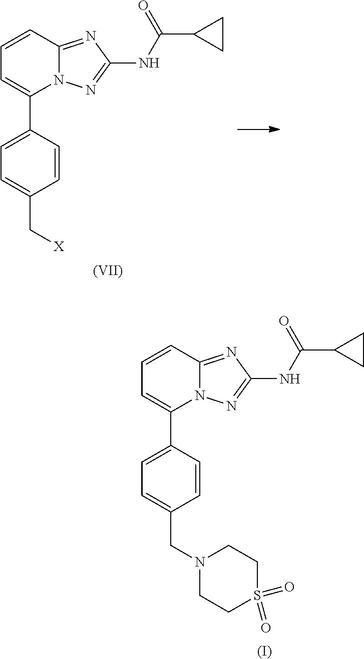

### DETAILED DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written

The description that follows, and the embodiments described herein, is provided by way of illustration of an example, or examples, of particular embodiments of the principles and aspects of the present disclosure. These examples are provided for the purposes of explanation, and not of limitation, of those principles and of the disclosure.

It should also be appreciated that the present disclosure can be implemented in numerous ways, including as a system, a method or a device. In this specification, these implementations, or any other form that the invention may take, may be referred to as processes. In general, the order of the steps of the disclosed processes may be altered within the scope of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

In an embodiment, the present invention related to a process for preparation of filgotinib compound of formula (I) or salts thereof comprising steps of;

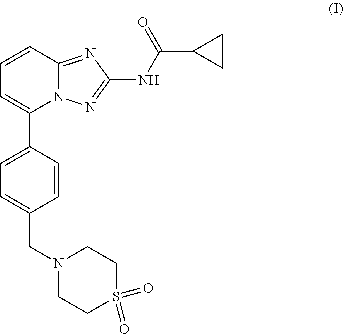 [SMILES: O=C(Nc1nc2cccc(-c3ccc(CN4CCS(=O)(=O)CC4)cc3)n2n1)C1CC1]

a) condensing compound of formula (II) or salt thereof with compound of formula (III) to obtain compound of formula (IV); wherein R is C₁-C₄ alkyl group;

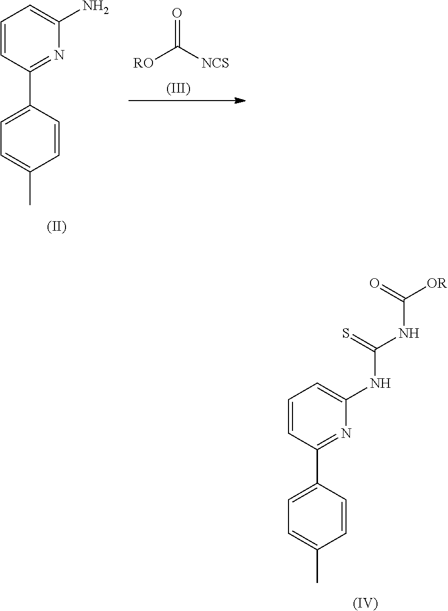

b) cyclizing compound of formula (IV) using hydroxyl amine or its acid additional salt to obtain compound of formula (V);

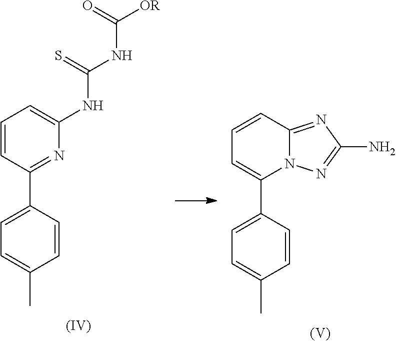

c) amidation of compound of formula (V) with cyclopropanecarbonyl chloride to obtain compound of formula (VI);

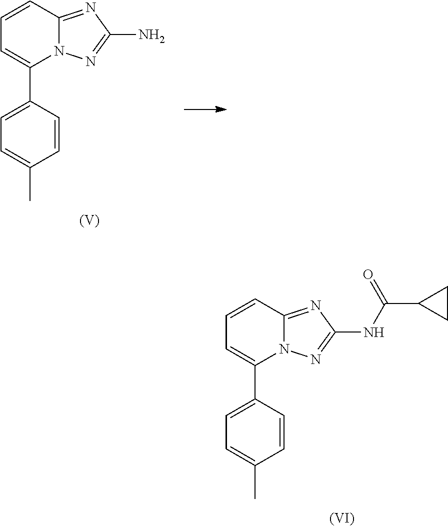 [SMILES: Cc1ccc(-c2cccc3nc(N)nn23)cc1 ; "(V)" ; Cc1ccc(-c2cccc3nc(NC(=O)C4CC4)nn23)cc1 ; "(VI)"]

d) treating compound of formula (VI) with halogenating agent to obtain compound of formula (VII);

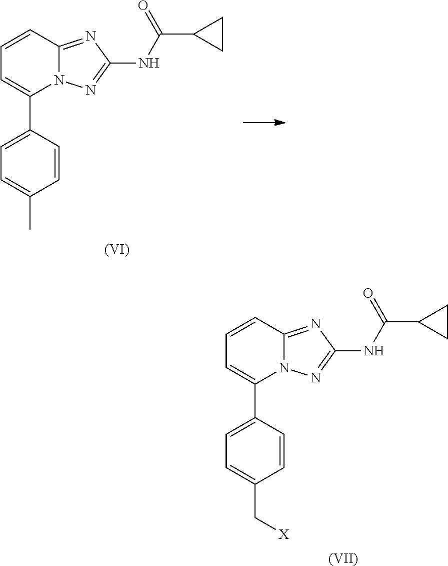

Wherein, X is Cl, Br, I or F.
e) condensing compound of formula (VII) with thiomorpholine-1,1-dioxide to obtain compound of formula (I);

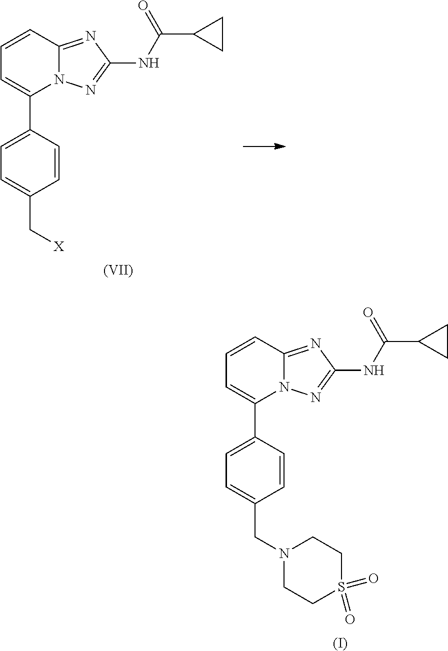

f) optionally converting compound of formula (I) into its salt.
   Stage (a): The condensation at stage (a) is performed in presence of organic solvent such as methylene chloride, ethylene chloride, THF, di-isopropyl ether or mixture(s) thereof. The reaction is carried out about 15 minutes to about 2 hours at ambient temperature.

The example of acid salts of compound of formula (II) includes hydrochloride, hydrobromide, oxalate, fumarate, tartarate and sulphate.

The term C₁-C₄ alkyl group used in the definition of “R” for compound of formula (II) includes methyl, ethyl, n-propyl, isopropyl, n-butyl, isobutyl and tert-butyl.

Isolation of compound of formula (IV) or its salt can be carried out by any method known in the art such as cooling, filtration, centrifugation, washing, drying and combination thereof.

Stage (b): The cyclization at stage (b) is performed in presence of alcoholic solvent and base at reflux temperature for about 1 to 5 hours.

The alcoholic solvent examples including but not limited to methanol, ethanol, propanol or mixture(s) thereof.

The base use for cyclization stage includes N,N-diisopropylethylamine, N,N-diisopropylamine.

The hydroxylamine or acid addition salts as used herein refers to acid addition salts of hydroxyl amine. Example includes without limitation hydroxylamine hydrochloride, hydroxylamine sulphate, hydroxylamine phosphate, and hydroxylamine nitrate.

Isolation of compound of formula (V) or its salt can be carried out by any method known in the art such as cooling, filtration, centrifugation, washing, drying and combination thereof.

Stage (c): The amidation at stage (c) is performed in presence of solvent such as methylene chloride, ethylene chloride, THF, di-isopropyl ether or mixture(s) thereof.

The base used for amidation is selected from N,N-Diisopropylethylamine (DIPEA), Triethylamine (TEA), N,N-diisopropylamine.

Isolation of compound of formula (VI) or its salt can be carried out by any method known in the art such as cooling, filtration, centrifugation, washing, drying and combination thereof.

Stage (d): The halogenation at stage (d) is performed in presence of solvent such as monochlorobenzene, toluene, acetonitrile, ethylene chloride, CCl4 or mixture(s) thereof.

Compound of formula (VI) is halogenated using brominating agent such as pyridinium tribromide, pyridinium dichlorobromate, 1,3-dibromo-5,5-dimethylhydantoin (DBDMH), tetrabromocyclohexadienone, N-Bromosuccinimide (NBS), tetraoctyl ammonium bromide (TOABr).

Compound of formula (VI) is halogenated using chlorinating agent such as Thionyl Chloride, Methanesulfonyl Chloride, Trichloromethanesulfonyl Chloride, tert-Butyl Hypochlorite, Dichloromethyl Methyl Ether, Methoxyacetyl Chloride, Oxalyl Chloride, Cyanuric Chloride, N-Chloro succinimide, N-Chlorophthalimide, 1,3-Dichloro5,5-dimethylhydantoin, Sodium Dichloroisocyanurate, Trichloroisocyanuric Acid, Chloramine B Hydrate, Dichloramine B, Dichloramine T, Benzyltrimethylammonium Tetrachloroiodate, Trimethylsilyl Chloride.

Compound of formula (VI) is halogenated using iodinating agent such as Iodine, Hydriodic Acid, Carbon Tetraiodide, 1-Chloro-2-iodoethane, N,N-Dimethyl-N-(methylsulfanylmethylene)-ammonium Iodide, N-Iodosuccinimide, N-Iodosaccharin, 1,3-Diiodo-5,5-dimethylhydantoin, Pyridine Iodine Monochloride, Tetramethylammonium Dichloroiodate, Benzyltrimethylammonium Dichloroiodate, B is(pyridine)iodonium Tetrafluoroborate, Bis(2,4,6-trimethylpyridine)-iodonium Hexafluorophosphate, Trimethylsilyl Iodide.

Compound of formula (VI) is halogenated using fluorinating agent such as Potassium Hydrogenfluoride, Tetramethylammonium Fluoride Tetrahydrate, Tetrabutylammonium Fluoride Hydrate, Tetrabutylammonium Fluoride, Triethylamine Trihydrofluoride, DMPU-HF Reagent, Tetraethylammonium Fluoride Trihydrofluoride, Tetrabutylammonium Bifluoride, 2-Fluoro-1-methylpyridinium p-Toluenesulfonate, DAST, Bis(2-methoxyethyl)-aminosulfur Trifluoride, Ishikawa's Reagent, PyFluor, Pyrimidine-2-sulfonyl Fluoride, Tetrabutylammonium Difluorotriphenylsilicate, Tetrabutylammonium Difluorotriphenylstannate, 1-Fluoropyridinium Trifluoromethanesulfonate, 1-Fluoropyridinium Tetrafluoroborate, 1-Fluoro-2,4,6-trimethylpyridinium Tetrafluoroborate, 1-Fluoro-2,6-dichloropyridinium Tetrafluoroborate, 1,1′-Difluoro-2,2′-bipyridinium Bis(tetrafluoroborate), N-Fluorobenzenesulfonimide, 1-Fluoro-3,3-dimethyl1,2-benziodoxole.

Halogenation reaction is further carried out in presence of radical initiator selected from AIBN [2,2′-Azobis(2-methylpropionitrile)], BPO [benzoyl peroxide].

Isolation of compound of formula (VII) or its salt can be carried out by any method known in the art such as cooling, filtration, centrifugation, washing, drying and combination thereof.

Stage (e): Condensation reaction of compound of formula (VII) and thiomorpholine-1,1-dioxide is performed in presence of organic solvent at room temperature for 20 to 48 hours to obtain compound of formula (I), which is optionally converted into its salt.

The organic solvent is selected from the solvent such as methanol, ethanol, isopropanol, n-propanol, tert-butanol, n-butanol or mixture(s) thereof.

Free base of compound of formula (I) is optionally isolate in organic solvent such as methanol, ethyl acetate, acetone, methyl ethyl ketone, methyl isobutyl ketone, tert-butanol, n-butanol, water or mixture(s) thereof.

Compound of formula (I) is optionally converted into its pharmaceutically acceptable salt in presence of solvent such as MDC, acetonitrile, 2-Methyl THF, iso-propoxy ethanol, 2-butoxy ethanol, iso-butyl alcohol, tert-butyl acetate or mixture(s) thereof at room temperature.

The term used “salt” or “pharmaceutically acceptable salt” in relation with formula (I), (IV) or (V) is refers to salt of compound of formula (I), (IV) or (V) that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

Yet another embodiment, the present invention relates to a process for preparation of compound of formula (IV) comprising condensing compound of formula (II) or salt thereof with compound of formula (III) to obtain compound of formula (IV); wherein R is C₁-C₄ alkyl group.

Reaction condition for the preparation of compound of formula (IV) is defined as above.

Yet another embodiment, the present invention relates to a process for preparation of compound of formula (V) comprising step of;
a) condensing compound of formula (II) or salt thereof with compound of formula (III) to obtain compound of formula (IV); wherein R is C₁-C₄ alkyl group;
b) cyclizing compound of formula (IV) using hydroxyl amine or its acid additional salt to obtain compound of formula (V).

Reaction condition for the preparation of compound of formula (V) is defined as above.

In another aspect, the present invention provides a novel intermediates compound of formula (IV) wherein R is C₁-C₄ alkyl group and compound of formula (V).

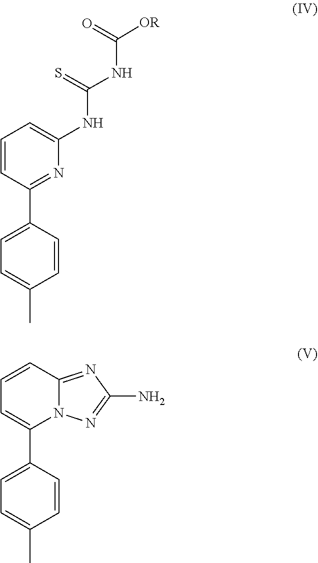

The following examples are presented for illustration only, and are not intended to limit the scope of the invention or appended claims

### Example 1: Preparation of 6-para-tolylpyridine-2-amine hydrochloride (Formula-IIa)

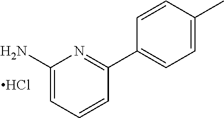

Triethylsilane (13.04 g, 0.112 mol) was added to the mixture of THF (60.0 ml), 4-(methylthio)-6-p-tolylpyridin-2-amine (10.0 g, 0.0374 mol) and 10% Pd/C(1.0 g) paste at 0° C. After addition the reaction mass was stirred at 0° C. for 30 min and then at room temperature for 3 h. The reaction mass was filtered through Celite bed and THF was distilled off to get oily residue. The oliy residue thus obtained was treated with methanolic hydrochloric acid solution to get 6-para-tolylpyridine-2-amine hydrochloride (Yield: 7.93 g, 95.88%). ¹H-NMR (400 MHz, DMSO) δ 14.13 (1H, br s), 8.34 (2H, br s), 7.94 (1H, dd J1 8.7 Hz, J2 7.4 Hz), 7.88 (2H, d J1 8.1 Hz), 7.39 (2H, d J1 8.1 Hz), 7.21 (1H, d, J1 7.4 Hz) 6.95 (1H, d, J1 8.1 Hz) 2.39 (3H, s).

### Example 2: Preparation of Ethyl amino-N-(6-p-tolylpyridin-2-yl) methanethiocarbamate (Formula-IVa)

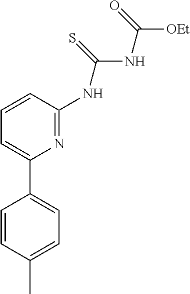 [SMILES: CCOC(=O)NC(=S)Nc1cccc(-c2ccc(C)cc2)n1]

To a solution of 6-para-tolylpyridine-2-amine (free base) (10.0 g, 0.054 mol) in DCM (100 mL) cooled to 5° C. was added ethoxycarbonyl isothiocyanate (17.3 mL, 0.062 mol) drop wise over 15 min. The reaction mixture was then allowed to warm to room temp. (20° C.) and stirred for 2 h. Product was collected by filtration under vacuum, thoroughly washed with DCM (2×10 mL) and air-dried to afford the desired product (Yield: 11.65 g, 81.58%). The thiourea derivative obtained was used as such for the next step. ¹H-NMR (400 MHz, DMSO) δ 11.91 (2H, br s), 8.66 (1H, s), 7.94 (3H, t), 7.77 (1H, dt), 7.31 (2H, d), 4.24 (2H, q), 2.36 (3H, s), 1.29 (3H, t).

### Example 3: Preparation of 5-p-tolyl-[1,2,4]triazolo[1,5-a]pyridin-2-amine (Formula-V)

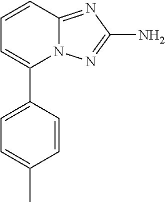 [SMILES: Cc1ccc(-c2cccc3nc(N)nn23)cc1]

To a suspension of hydroxylamine hydrochloride (10.94 g, 0.157 mol) in mixture of EtOH:MeOH (1:1, 100 mL) was added N,N′-diisopropylethylamine (16.19 mL, 0.093 mol) and the mixture was stirred at room temp. (20° C.) for 1 h. Ethyl amino-N-(6-p-tolylpyridin-2-yl) methane thio carbamate (Formula IVa) (10.0 g, 0.031 mol) was then added and the mixture slowly heated to reflux (Note: bleach scrubber was required to quench H₂S evolved). After 3 h at reflux, the mixture was allowed to cool and evaporated in vacuum, addition of H₂O (100 mL) into oily residue and stirred for 1 h. The solid precipitates was filtered and washed successively with H₂O (50 mL) then dried in vacuum to afford the triazolo pyridine derivative (Formula V) as a solid (Yield: 7.13 g, 86.9%). ¹H-NMR (400 MHz, DMSO-d4) δ 7.81 (2H, d, J 8.1, a 2× aromatic-H), 7.45 (1H, t, J 8.1 Hz, aromatic-H), 7.29 (3H, m), 6.94 (1H, d J 7.4 Hz), 6.00 (2H, br, S, NH₂), 2.34 (3H, S).

### Example 4: Preparation of N-(5-p-tolyl-[1,2,4]triazolo[1,5,a]pyridin2-yl)cyclopropane carboxamide (Formula-VI)

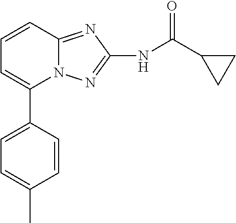 [SMILES: Cc1ccc(-c2cccc3nc(NC(=O)C4CC4)nn23)cc1]

To a solution of the 5-p-tolyl-[1,2,4]triazolo[1,5-a]pyridin-2-amine (Formula-V) (10.0 g, 0.044 mol) in MDC (100 mL) at 25° C. was added DIPEA (38.24 mL, 0.22 mol) and stirred for 30 min. followed by addition of cyclopropanecarbonyl chloride (13.98 g, 0.133 mol). The reaction mixture was stirred at same temperature for 1 h. Followed by solvent evaporation in vacuum and the resultant residue was treated with methanolic ammonia solution (1000 mL) and stirred at ambient temp, (for 1-16 h) to hydrolyze any bis-acylated product. Product isolation was made by removal of solvent in vacuum followed by tituration with Et₂O (50 mL). The solids were collected by filtration, washed with H₂O (2×50 mL) dried in vacuum to give the desired compound of Formula VI. (Yield: 9.8 g, 75.21%). ¹H-NMR (400 MHz, DMSO) δ 11.04 (1H, S), 7.91 (2H, d, J 8.1 Hz, 2× aromatic-H), 7.68 (2H, m), 7.37 (2H, d J 8.1 Hz), 7.26 (1H, dd J1 6.7 Hz, J2 2.0 Hz), 2.40 (3H S), 2.01 (1H, S), 0.81 (3H d).

### Example 5: Preparation of Cyclopropanecarboxylic acid [5-(4-bromomethyl-phenyl)-[1,2,4]triazolo[1,5-a]pyridin-2-yl]-amide (Formula-VIIa)

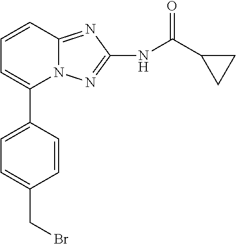 [SMILES: O=C(Nc1nc2cccc(-c3ccc(CBr)cc3)n2n1)C1CC1]

To the solution of N-(5-p-tolyl-[1,2,4]triazolo[1,5,a]pyridin2-yl)cyclopropane carboxamide (10.0 g, 0.034 mol) in mono chloro benzene (300 mL), N-bromo succinamide (6.08 g, 0.034 mol) and AIBN (0.558 g, 0.0034 mol) was added. The resulting mixture was heated at 70° C. for 4 h. The reaction mass quenched in water and extracted with ethyl acetate (300 mL×2). The organic layer was washed with sodium thiosulphate solution (200 mL). The ethyl acetate was distilled under vacuum and degas. Charged acetone (50 mL) to the degas mass, stirred, filtered and washed with acetone (10 mL) to get Formula VII (Yield: 7.96 g, 62.74%). ¹H-NMR (400 MHz, DMSO) δ 11.07 (1H, br s), 8.03 (2H, m), 7.91 (1H, d), 7.73 (3H, m), 7.64 (2H, m), 7.34 (2H, m), 4.81 (2H, s) 2.01 (1H, s), 0.81 (4H, d).

### Example 6: Preparation of N-[5-[4-[(1,1-dioxo-1,4-thiazinan-4-yl)methyl]phenyl]-[1,2,4]-triazolo[1,5-a]-pyridin-2-yl]-cyclopropane carboxamide (Filgotinib) (Formula I)

Cyclopropanecarboxylic acid [5-(4-bromomethyl-phenyl)-[1,2,4]triazolo[1,5-a]pyridin-2-yl]-amide (10.0 g, 0.026 mol) and DIPEA (18.73 mL, 0.107 mol) were dissolved in DCM/MeOH (5:1, v:v) under N₂ and thiomorpholine 1,1-dioxide (4.63 g 0.0269 mol) was added. The resulting solution was stirred at room temperature till completion. After the reaction completion solvent was evaporated. The oily residue thus obtained was treated with ethyl acetate and methanol to afford N-[5-[4-[(1,1-dioxo-1,4-thiazinan-4-yl)methyl]phenyl]-[1,2,4]-triazolo[1,5-a]pyridin-2-yl]cyclopropane carboxamide (Filgotinib Free base, Formula I) (Yield: 5.87 g, 51.28%). ¹H-NMR (400 MHz, DMSO) δ 11.06 (1H, s), 7.99 (2H, dJ1 8.1 Hz), 7.70 (2H, m), 7.52 (2H, d J1 8.7 Hz), 7.30 (1H, dd J1 6.5 Hz, J2 1.2 Hz), 3.77 (2H, s) 3.15 (4H, t, J1 4.7 Hz) 2.94 (4H, d J1 2.7 Hz), 2.00 (1H, s), 0.81 (4H, d, J1 6.0 Hz).

### Example 7: Preparation of N-[5-[4-[(1,1-dioxo-1,4-thiazinan-4-yl)methyl] phenyl]-[1,2,4]-triazolo[1,5-a]pyridin-2-yl]cyclopropane carboxamide (Filgotinib) (Formula I)

Cyclopropanecarboxylic acid [5-(4-bromomethyl-phenyl)-[1,2,4]triazolo[1,5-a]pyridin-2-yl]-amide (10.0 g, 0.026 mol) and DIPEA (18.73 mL, 0.107 mol) were dissolved in DCM/MeOH (5:1,v:v) under N₂ and thiomorpholine 1,1-dioxide (4.63 g 0.0269 mol) was added. The resulting solution was stirred at room temperature till completion. After the reaction completion solvent was evaporated. The oily residue thus obtained was treated with water and the product collected by filtration. The wet cake thus obtained was treated with ethyl acetate and methanol to afford N-[5-[4-[(1,1-dioxo-1,4-thiazinan-4-yl)methyl]-phenyl]-[1,2,4]-triazolo[1,5-a] pyridin-2-yl]cyclopropane carboxamide (Filgotinib Free base, Formula I).

### Example 8: Preparation of N-[5-[4-[(1,1-dioxo-1,4-thiazinan-4-yl)methyl] phenyl]-[1,2,4]-triazolo[1,5-a]pyridin-2-yl]cyclopropane carboxamide (Filgotinib) (Formula I)

Cyclopropanecarboxylic acid [5-(4-bromomethyl-phenyl)-[1,2,4]triazolo[1,5-a]pyridin-2-yl]-amide (10.0 g, 0.026 mol) and DIPEA (18.73 mL, 0.107 mol) were dissolved in DCM/MeOH (5:1,v:v) under N₂ and thiomorpholine 1,1-dioxide (4.63 g 0.0269 mol) was added. The resulting solution was stirred at room temperature till completion. After the reaction completion solvent was evaporated. The oily residue thus obtained was taken in MDC followed by water washing and evaporation. The oily residue thus obtained was treated with ethyl acetate and methanol to afford N-[5-[4-[(1,1-dioxo-1,4-thiazinan-4-yl) methyl]-phenyl]-[1,2,4]-triazolo[1,5-a] pyridin-2-yl]cyclo propane carboxamide (Filgotinib Free base, Formula I).

### Example 9: Preparation of N-[5-[4-[(1,1-dioxo-1,4-thiazinan-4-yl)methyl]phenyl]-[1,2,4]-triazolo[1,5-a]pyridin-2-yl]cyclopropane carboxamide hydrochloride (Filgotinib HCl)

2.5 gm of N-[5-[4-[(1,1-dioxo-1,4-thiazinan-4-yl)methyl]phenyl]-[1,2,4]-triazolo[1,5-a]pyridin-2-yl]cyclopropane carboxamide (i.e. Filgotinib free base) was dissolved in mixture of 87.5 ml of MDC and 43.75 mL acetonitrile. While stirring at room temperature 5 mL of 12% methanolic hydrochloride solution was added drop-wise. A white precipitate was formed and mixture was stirred for 30-90 min at room temperature. The white product was filtered and washed with mixture of MDC and acetonitrile to get filgotinib hydrochloride salt.

A skilled artisan will appreciate that the quantity and type of each ingredient can be used in different combinations or singly. All such variations and combinations would be falling within the scope of present disclosure.

The foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

1. A process for preparation of a compound of formula (I), or a pharmaceutically acceptable salt thereof, comprising the steps of: 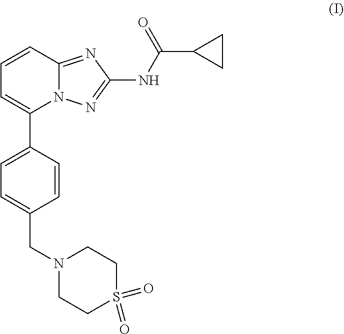 [SMILES: O=C(Nc1nc2cccc(-c3ccc(CN4CCS(=O)(=O)CC4)cc3)n2n1)C1CC1]
a) condensing a compound of formula (II) or salt thereof with a compound of formula (III) in organic solvent to obtain a compound of formula (IV); wherein R is C₁-C₄ alkyl; 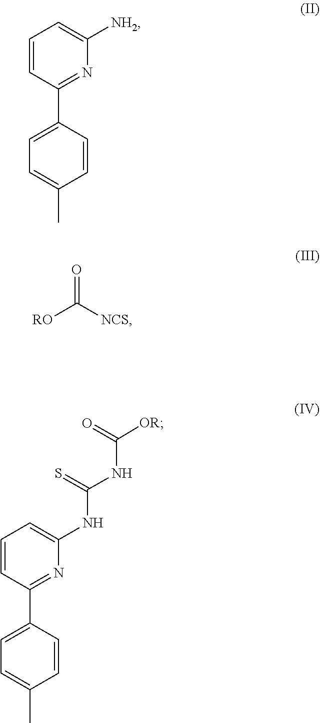
b) cyclizing the compound of formula (IV) using hydroxyl amine or its acid additional salt in presence of a solvent and a base to obtain a compound of formula (V) or a salt thereof; 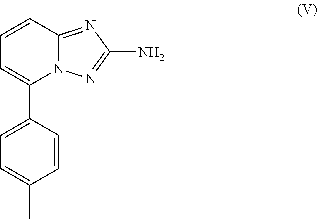 [SMILES: Cc1ccc(-c2cccc3nc(N)nn23)cc1]
c) reacting the compound of formula (V) with cyclopropanecarbonyl chloride in presence of a solvent and a base to obtain a compound of formula (VI); 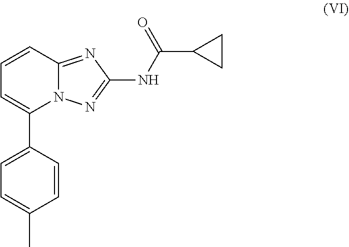 [SMILES: Cc1ccc(-c2cccc3nc(NC(=O)C4CC4)nn23)cc1]
d) treating the compound of formula (VI) with halogenating agent in presence of a solvent to obtain a compound of formula (VII); 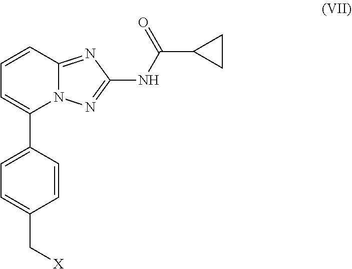
wherein, X is Cl, Br, I or F;
e) condensing the compound of formula (VII) with thiomorpholine-1,1-dioxide in presence of organic solvent to obtain a compound of formula (I); and
f) optionally converting the compound of formula (I) into its pharmaceutically acceptable salt.

2. The process as claimed in claim 1, wherein the organic solvent in stage a) is selected from methylene chloride, ethylene chloride, tetrahydrofuran, di-isopropyl ether or mixture(s) thereof.

3. The process as claimed in claim 1, wherein the solvent in stage b) and stage c) is selected from alcohol, methylene chloride, ethylene chloride, THF, di-isopropyl ether, or mixture(s) thereof and the base in stage b) and stage c) is selected from N,N-diisopropylethylamine, triethylamine, and N,N-diisopropylamine.

4. The process as claimed in claim 1, wherein the halogenating agent is selected from pyridinium tribromide, pyridinium dichlorobromate, 1,3-dibromo-5,5-dimethylhydantoin (DBDMH), tetrabromocyclohexadienone, N-bromosuccinimide (NBS), tetraoctyl ammonium bromide (TOABr), thionyl chloride, methanesulfonyl chloride, trichloromethanesulfonyl chloride, tert-butyl hypochlorite, dichloromethyl methyl ether, methoxyacetyl chloride, oxalyl chloride, cyanuric chloride, N-chlorosuccinimide, N-chlorophthalimide, 1,3-dichloro5,5-dimethylhydantoin, sodium dichloroisocyanurate, trichloroisocyanuric acid, chloramine B hydrate, dichloramine B, dichloramine T, benzyltrimethylammonium tetrachloroiodate, trimethylsilyl chloride, iodine, hydriodic acid, carbon tetraiodide, 1-chloro-2-iodoethane, n,n-dimethyl-n-(methylsulfanylmethylene)-ammonium iodide, n-iodosuccinimide, n-iodosaccharin, 1,3-diiodo-5,5-dimethylhydantoin, pyridine iodine monochloride, tetramethylammonium dichloroiodate, benzyltrimethylammonium dichloroiodate, bis(pyridine)iodonium tetrafluoroborate, bis(2,4,6-trimethylpyridine)-iodonium hexafluorophosphate, trimethylsilyl iodide, potassium hydrogenfluoride, tetramethylammonium fluoride tetrahydrate, tetrabutylammonium fluoride hydrate, tetrabutylammonium fluoride, triethylamine trihydrofluoride, dmpu-hf reagent, tetraethylammonium fluoride trihydrofluoride, tetrabutylammonium bifluoride, 2-fluoro-1-methylpyridinium p-toluenesulfonate, DAST, bis(2-methoxyethyl)-aminosulfur trifluoride, ishikawa's reagent, pyfluor, pyrimidine-2-sulfonyl fluoride, tetrabutylammonium difluorotriphenylsilicate, tetrabutylammonium difluorotriphenylstannate, 1-fluoropyridinium trifluoromethanesulfonate, 1-fluoropyridinium tetrafluoroborate, 1-fluoro-2,4,6-trimethylpyridinium tetrafluoroborate, 1-fluoro-2,6-dichloropyridinium tetrafluoroborate, 1,1′-difluoro-2,2′-bipyridinium bis(tetrafluoroborate), N-fluorobenzenesulfonimide and 1-fluoro-3,3-dimethyl1,2-benziodoxole.

5. The process as claimed in claim 1, wherein the organic solvent in stage e) is selected from alcohol, methylene chloride, ethylene chloride, THF, di-isopropyl ether, and a mixture thereof.

6. The process as claimed in claim 3, wherein the alcohol is selected from methanol, ethanol, isopropanol, n-propanol, tert-butanol, n-butanol or mixture(s) thereof.

7. An intermediate compound formula (IV), (V) or its salt thereof, 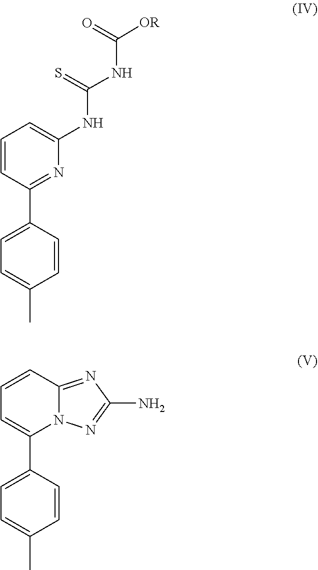
wherein R is C₁-C₄ alkyl.

8. A process for preparation of a intermediate compound of formula (IV) or its salt thereof comprising the step of condensing a compound of formula (II) or salt thereof with a compound of formula (III) in organic solvent to obtain a compound of formula (IV); wherein R is C₁-C₄ alkyl;

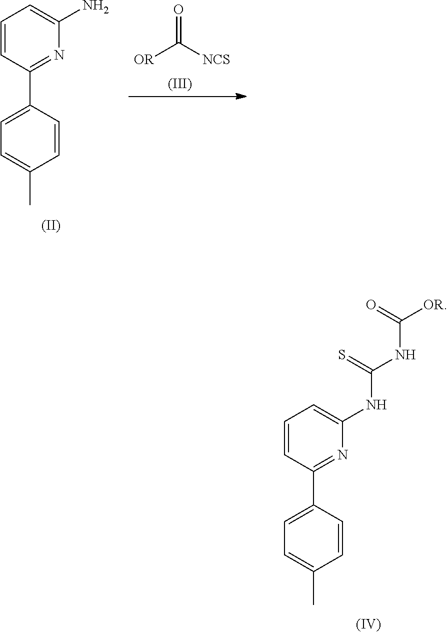

9. A process for preparation of intermediate compound of formula (V) or its salt thereof comprising the steps of:
a) condensing a compound of formula (II) or salt thereof with a compound of formula (III) in organic solvent to obtain a compound of formula (IV); wherein R is C₁-C₄ alkyl; and 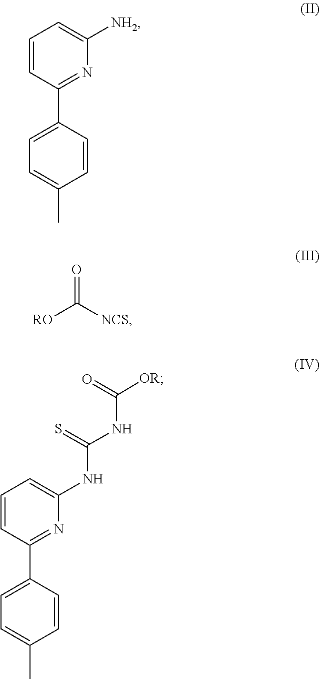
b) cyclizing the compound of formula (IV) using hydroxyl amine or its acid additional salt in presence of a solvent and a base to obtain a compound of formula (V) or a salt thereof;

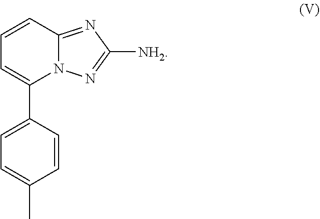 [SMILES: Cc1ccc(-c2cccc3nc(N)nn23)cc1]

10. The compound of formula (IV), (V) or its salt thereof for the preparation of a compound of formula (I) or a pharmaceutically acceptable salt thereof.

11. The process as claimed in claim 5, wherein the alcohol is selected from methanol, ethanol, isopropanol, n-propanol, tert-butanol, n-butanol or mixture(s) thereof.
